(19) United States
(12) Patent Application Publication
(10) Pub. No.: US 2022/0304921 A1
(43) Pub. Date: Sep. 29, 2022

(54) POUCHED PRODUCTS

(71) Applicant: NICOVENTURES TRADING LIMITED, London (GB)

(72) Inventors: David Neil McClanahan, Winston-Salem, NC (US); Dwayne William Beeson, Kernersville, NC (US); Laya Katina Palmer Horton, Winston-Salem, NC (US); Ronald K. Hutchens, East Bend, NC (US); Savannah Johnson, Winston-Salem, NC (US); Wesley Steven Jones, Lexington, NC (US); Travis O'Neal, Pinnacle, NC (US); Pankaj Patel, Clemmons, NC (US)

(21) Appl. No.: 17/836,827

(22) Filed: Jun. 9, 2022

Related U.S. Application Data
(63) Continuation of application No. PCT/IB2020/061411, filed on Dec. 2, 2020.
Application No. PCT/IB2020/061411 is a continuation-in-part of application No. 16/707,390, filed on Dec. 9, 2019.

(51) Int. Cl. A61K 9/00

(52) U.S. Cl. CPC A61K 9/009; A61K 9/0056

(57) ABSTRACT

The disclosure provides pouched products configured for oral use, including a material within a porous pouch, where the porous pouch has four sides of small dimension, where the porous pouch has four sides of large dimension, and/or wherein the porous pouch has a shape other than square or rectangular, e.g., a rounded shape. The disclosure further provides methods of providing a modified mouthfeel with respect to conventional pouched products by providing pouched product in such sizes and/or shapes.

### FIELD OF THE DISCLOSURE

The present disclosure relates to flavored products intended for human use. The products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

All-white snus portions are growing in popularity, and offer a discrete and aesthetically pleasing alternative to traditional snus. Such modern “white” pouched products may include a bleached tobacco or may be tobacco-free.

### BRIEF SUMMARY

The present disclosure generally provides products configured for oral use, and specifically provides such products with modified mouthfeel with respect to traditional pouched products. The products are intended to impart a taste when used orally, and typically also deliver active ingredients to the consumer, such as nicotine. The products and methods provided herein impart such taste and/or deliver such active ingredients via a pouched product that is smaller than conventional pouched products and/or a pouched product that has a modified shape as compared with conventional pouched products.

In one aspect is provided a pouched product for oral use, comprising a material within a porous pouch, wherein the material comprises one or more flavoring agents and/or one or more active ingredients; wherein the porous pouch comprises four sides, and wherein none of the four sides of the porous pouch is more than 24 mm in length. In some embodiments, none of the four sides of the porous pouch is more than 22 mm in length. In some embodiments, none of the four sides of the porous pouch is more than 20 mm in length. In some embodiments, the four sides have lengths of about 24 mm, about 24 mm, about 9 mm, and about 9 mm. In some embodiments, the four sides have lengths that are substantially the same. In some embodiments, the four sides have lengths of about 16 mm or less.

In another aspect, the disclosure provides a pouched product for oral use, comprising a material within a porous pouch, wherein the material comprises one or more flavoring agents and/or one or more active ingredients; wherein the porous pouch is in a shape that is not square or rectangular. In some embodiments, the shape is selected from the group consisting of triangular, rhombic, pentagonal, hexagonal, heptagonal, and octagonal (and such shapes with rounded corners). In some embodiments, the shape has one or more rounded sides and/or rounded corners (including all rounded sides and/or all rounded corners). In some embodiments, the shape is selected from the group consisting of circular, semi-circular, oval, semi-oval, kidney-shaped, teardrop-shaped, star-shaped, heart-shaped, and crescent-shaped.

In a further aspect is provided a method of delivering one or more flavoring agents and/or one or more active ingredients, comprising providing the one or more flavoring agents and/or one or more active ingredients in the form of a material within a porous pouch, wherein the porous pouch comprises four sides, and wherein none of the four sides of the porous pouch is more than 24 mm in length; or wherein the porous pouch is in a shape that is not square or rectangular.

The material within the porous pouch of the disclosed oral products can vary. In some embodiments, the material further comprises one or more particulate fillers and water. In some embodiments, such one or more particulate fillers comprise a cellulose material, e.g., microcrystalline cellulose. In some embodiments, the porous pouch comprises one or more active ingredients, wherein the one or more active ingredients are selected from the group consisting of a nicotine component, a botanical, a nutraceutical, a stimulant, an amino acid, a vitamin, a cannabinoid, a cannabimimetic, a terpene, and combinations thereof. In some embodiments, the porous pouch comprises one or more flavoring agents, wherein the one or more flavoring agents comprise one or more of ethyl vanillin, cinnamaldehyde, sabinene, limonene, gamma-terpinene, beta-farnesene, and citral.

The disclosure includes, without limitations, the following embodiments.

Embodiment 1: A pouched product for oral use, comprising a material within a porous pouch, wherein the material comprises one or more flavoring agents and/or one or more active ingredients; wherein the porous pouch comprises four sides, and wherein none of the four sides of the porous pouch is more than 24 mm in length.

Embodiment 2: The pouched product of Embodiment 1, wherein none of the four sides of the porous pouch is more than 22 mm in length.

Embodiment 3: The pouched product of any of Embodiments 1-2, wherein none of the four sides of the porous pouch is more than 20 mm in length.

Embodiment 4: The pouched product of any of Embodiments 1-3, wherein the four sides have lengths of about 24 mm, about 24 mm, about 9 mm, and about 9 mm.

Embodiment 5: The pouched product of any of Embodiments 1-4, wherein the four sides have lengths that are substantially the same.

Embodiment 6: The pouched product of any of Embodiments 1-5, wherein the four sides have lengths of about 16 mm or less, about 14 mm or less, about 12 mm or less, about 10 mm or less, or about 9 mm or less.

Embodiment 7: A pouched product for oral use, comprising a material within a porous pouch, wherein the material comprises one or more flavoring agents and/or one or more active ingredients; wherein the porous pouch is in a shape that is not square or rectangular.

Embodiment 8: The pouched product of Embodiment 7, wherein the shape is selected from the group consisting of triangular, rhombic, pentagonal, hexagonal, heptagonal, and octagonal.

Embodiment 9: The pouched product of any of Embodiments 1-8, wherein the shape of the porous pouch has one or more rounded sides.

Embodiment 10: The pouched product of any of Embodiments 7-9, wherein the shape of the porous pouch is selected from the group consisting of circular, semi-circular, oval, semi-oval, kidney-shaped, teardrop-shaped, star-shaped, heart-shaped, and crescent-shaped.

Embodiment 11: A pouched product for oral use, comprising a material within a porous pouch, wherein the material comprises one or more flavoring agents and/or one or more active ingredients; wherein the porous pouch comprises four sides, and wherein two of the four sides have a length of about 8 mm or greater and the other of the four sides have a length of about 35 mm or greater.

Embodiment 12: The pouched product of Embodiment 11, wherein two of the four sides have a length of about 10 mm or greater and the other of the four sides have a length of about 40 mm or greater.

Embodiment 13: The pouched product of any of Embodiments 11 and 12, wherein two of the four sides have a length of about 12 mm or greater and the other of the four sides have a length of about 40 mm or greater.

Embodiment 14: The pouched product of any of Embodiments 11-13, wherein two of the four sides have a length of about 10 mm or greater and the other of the four sides have a length of about 50 mm or greater.

Embodiment 15: The pouched product of any of Embodiments 11-14, wherein two of the four sides have a length of about 12 mm or greater and the other of the four sides have a length of about 50 mm or greater.

Embodiment 16: The pouched product of any of Embodiments 11-15, wherein two of the four sides have a length of about 8 to about 18 mm and the other of the four sides have a length of about 35 to about 60 mm.

Embodiment 17: The pouched product of any of Embodiments 1-16, wherein the material within the porous pouch further comprises one or more particulate fillers and water.

Embodiment 18: The pouched product of Embodiment 17, wherein the one or more particulate fillers comprise a cellulose material.

Embodiment 19: The pouched product of Embodiment 18, wherein the cellulose material comprises microcrystalline cellulose.

Embodiment 20: The pouched product of any of Embodiments 17-19, wherein the one or more particulate fillers further comprise a cellulose derivative in an amount by weight of the material of from about 1% to about 3%.

Embodiment 21: The pouched product of any of Embodiments 1-20, wherein the one or more active ingredients are selected from the group consisting of a nicotine component, a botanical, a nutraceutical, a stimulant, an amino acid, a vitamin, a cannabinoid, a cannabimimetic, a terpene, and combinations thereof.

Embodiment 22: The pouched product of any of Embodiments 1-21, wherein the material comprises from about 0.001 to about 10% by weight of a nicotine component, calculated as the free base.

Embodiment 23: The pouched product of any of Embodiments 1-22, wherein the material comprises no more than about 10% by weight of a tobacco material, excluding any nicotine component present.

Embodiment 24: The pouched product of any of Embodiments 1-22, wherein the material is substantially free of tobacco material.

Embodiment 25: The pouched product of any of Embodiments 1-24, wherein the material is substantially free of tobacco material, excluding any nicotine component present.

Embodiment 26: The pouched product of any of Embodiments 1-25, wherein the one or more flavoring agents comprises one or more of ethyl vanillin, cinnamaldehyde, sabinene, limonene, gamma-terpinene, beta-farnesene, and citral.

Embodiment 27: The pouched product of any of Embodiments 1-26 wherein the one or more flavoring agents comprises a compound having a carbon-carbon double bond, a carbon-oxygen double bond, or both.

Embodiment 28: The pouched product of any of Embodiments 1-27, wherein the one or more flavoring agents comprises one or more aldehydes, ketones, esters, terpenes, terpenoids, trigeminal sensates, or a combination thereof.

Embodiment 29: The pouched product of any of Embodiments 1-28, wherein the one or more flavoring agents comprises ethyl vanillin

Embodiment 30: The pouched product of any of Embodiments 1-29, wherein the material further comprises one or more salts, one or more sweeteners, one or more binding agents, one or more humectants, one or more gums, one or more active ingredients, a tobacco material, or combinations thereof.

Embodiment 31: The pouched product of any of Embodiments 1-30, wherein the size and/or shape of the porous pouch is adapted to fit comfortably within the oral cavity of a consumer.

Embodiment 32: The pouched product of any of Embodiments 1-31, wherein the size and/or shape of the porous pouch is designed to conform to a consumer's jaw size and/or shape or a consumer's gumline size and/or shape.

Embodiment 33: The pouched product of any of Embodiments 1-32, wherein the pouched product has improved mouthfeel as compared with conventional pouched products (e.g., products with larger dimensions and/or with square/rectangular shapes).

Embodiment 34: The pouched product of any of Embodiments 1-33, wherein the pouched product is substantially free of a tobacco material.

Embodiment 35: A method of providing a product with improved mouthfeel, comprising providing the pouched product of any of Embodiments 1-34 for use within the oral cavity.

Embodiment 36: A method of delivering one or more flavoring agents and/or one or more active ingredients, comprising providing the one or more flavoring agents and/or one or more active ingredients in the form of the pouched product of any of Embodiments 1-34.

Embodiment 37: Use of the pouched product of any of Embodiments 1-34 to provide improved mouthfeel to a user.

Embodiment 38: Use of the pouched product of any of Embodiments 1-34 to deliver one or more flavoring agents and/or one or more active ingredients to a user.

Embodiment 39: A method of delivering one or more flavoring agents and/or one or more active ingredients, comprising providing the one or more flavoring agents and/or one or more active ingredients in the form of a material within a porous pouch, wherein the porous pouch comprises four sides, and wherein: none of the four sides of the porous pouch is more than 24 mm in length (e.g., having any of the sizes referenced in Embodiments 1-6); two of the four sides have a length of about 8 mm or greater and the other of the four sides have a length of about 35 mm or greater (e.g., having any of the sizes referenced in Embodiments 11-16); or wherein the porous pouch is in a shape that is not square or rectangular (e.g., any of the shapes referenced in Embodiments 7-10).

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure.

FIG. 1 is a perspective view of one embodiment of a pouched product, taken across the width of the product, showing a rectangular outer pouch filled with a mixture;

FIG. 2 is a perspective view of another embodiment of a pouched product, taken across the width of the product, showing a squared outer pouch filled with a mixture;

FIGS. 3A, 3B, 3C, 3D, 3E, 3F, 3G, and 3H are example shapes of various embodiments of pouched products disclosed herein; and

FIGS. 4A, 4B, 4C, 4D, 4E, and 4F are further example shapes of various embodiments of pouched products disclosed herein.

### DETAILED DESCRIPTION

The present disclosure provides products with modified physical properties and modified appearance, e.g., modified size and/or shape. For consumer satisfaction, it is desirable to provide products adapted for oral use which exhibit suitable mouthfeel. With variations in the size and shape of consumer's oral cavities and variations in their preferences with respect to use and feel of a product within their oral cavity, the disclosure provides a range of products physically adapted to accommodate such variations. For example, the disclosure provides, in some embodiments, physically smaller products, e.g., to allow the consumer to move the product more freely around the oral cavity. The disclosure also provides, in some embodiments, physically larger products. The disclosure also provides, in some embodiments, products of varying shape, which may provide for a modified mouthfeel (e.g., enhanced smoothness within and/or conformity to the oral cavity).

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a mixture reflects the total wet weight of the mixture (i.e., including water).

The disclosure generally provides products configured for oral use. The term “configured for oral use” as used herein means that the product is provided in a form such that during use, saliva in the mouth of the user causes one or more of the components of the mixture (e.g., flavoring agents and/or active ingredient(s)) to pass into the mouth of the user. In certain embodiments, the product is adapted to deliver components to a user through mucous membranes in the user's mouth and, in some instances, said component is an active ingredient (including, but not limited to, for example, nicotine) that can be absorbed through the mucous membranes in the mouth when the product is used.

In particular, the disclosure provides products in the form of a mixture of one or more components, disposed within a moisture-permeable container (e.g., a water-permeable pouch). Such mixtures in the water-permeable pouch format are typically used by placing a pouch containing the mixture in the mouth of a human subject/user. Generally, the pouch is placed somewhere in the oral cavity of the user, for example under the lips, in the same way as moist snuff products are generally used. The pouch preferably is not swallowed. Exposure to saliva then causes some of the components of the mixture therein (e.g., flavoring agents and/or active ingredient) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been absorbed through oral mucosa of the human subject, and the pouch may be removed from the mouth of the consumer for disposal.

Certain embodiments of the disclosure will be described with reference to the figures of the accompanying drawings, and these described embodiments involve snus-type products having an outer pouch and containing a mixture of components (as referenced herein below). The pouched product 100 includes a moisture-permeable container in the form of a pouch 102, which contains a material 104 comprising a mixture of components. As explained in greater detail below, such embodiments are provided by way of example only. In particular, the size and shape of the illustrated outer pouches can vary as described in detail herein. The mixture/construction of such packets or pouches, such as the container pouch 102 in the embodiment illustrated in the figures, may be varied.

Suitable materials for the packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The mixture may be contained in pouches and packaged, in a manner and using the types of components used for the manufacture of conventional snus types of products. The pouch is typically a porous pouch, which is a liquid-permeable container of a type that may be considered to be similar in character to the mesh-like type of material that is used for the construction of a tea bag. Components of the mixture readily diffuse through the pouch and into the mouth of the user. Non-limiting examples of pouches are set forth in, for example, U.S. Pat. No. 5,167,244 to Kjerstad and 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which are incorporated herein by reference. As provided herein, such example pouches are considered herein to be “conventional” products, which are provided as comparisons to the pouches disclosed herein, which exhibit various modifications with respect to one or more such conventional products. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and mixture each may undergo complete dispersion within the mouth of the user during normal conditions of use, and hence the pouch and mixture both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the mixture contents permeate through the pouch material prior to the time that the pouch undergoes loss of its physical integrity. If desired, flavoring ingredients, disintegration aids, and other desired components, may be incorporated within, or applied to, the pouch material.

### Nano Pouches

The products described herein can, in some embodiments, be described as “nano pouches.” A “nano pouch” or “nano pouch product” as provided herein is a pouch with a size that is below that of conventional pouched products. Advantageously, all dimensions of such nano pouches are relatively small, e.g., with no dimension greater than about 30 mm (including no dimension greater than 30 mm) In some embodiments, no dimension of the nano pouches provided herein is greater than 30 mm, no dimension is greater than 28 mm, no dimension is greater than 26 mm, no dimension is greater than 24 mm, no dimension is greater than 22 nm, no dimension is greater than 20 nm, no dimension is greater than 18 nm, no dimension is greater than 16 nm, no dimension is greater than 14 nm, no dimension is greater than 12 nm, or no dimension is greater than 10 nm.

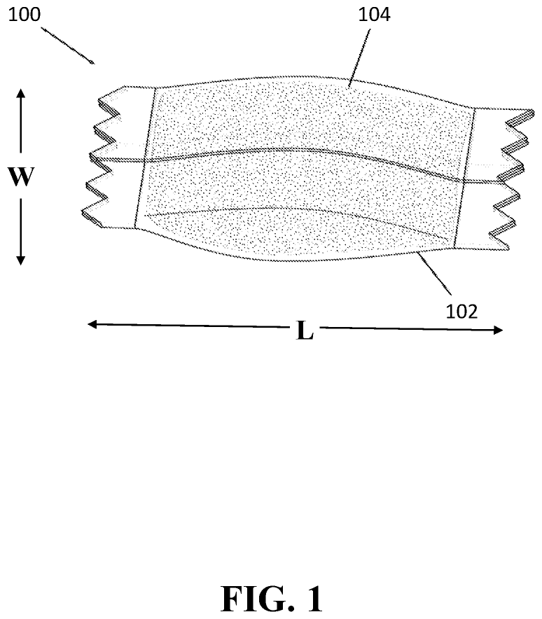

The sizing of certain pouched products according to the present disclosure can be understood through reference to FIG. 1, wherein “L” is used to refer to the length of the pouch, and “W” is used to refer to the width of the pouch. These pouches are referred to herein as “rectangular,” although the edges, as shown in FIG. 1, may be rounded to some extent. Certain, non-limiting examples of rectangular nano pouches provided herein are as follows: a nano pouch with L≤30 mm and W≤10 mm, a nano pouch with L≤30 mm and W≤9 mm, a nano pouch with L≤28 mm and W≤10 mm, a nano pouch with L≤28 mm and W≤9 mm, a nano pouch with L≤26 mm and W≤10 mm, a nano pouch with L≤26 mm and W≤9 mm, a nano pouch with L≤24 mm and W≤10 mm, and a nano pouch with L≤24 mm and W≤9 mm. Certain advantageous ranges of length and width are, in some embodiments, a length L of about 16 mm to about 30 mm, such as about 16 mm to about 30 mm, about 16 mm to about 28 mm, about 16 mm to about 26 mm, about 16 mm to about 24 mm, about 16 mm to about 22 nm, about 16 mm to about 20 nm, about 18 mm to about 30 mm, about 18 mm to about 28 mm, about 18 mm to about 26 mm, about 18 mm to about 24 mm, about 18 mm to about 22 nm, about 18 mm to about 20 nm, about 20 mm to about 30 mm, about 20 mm to about 28 mm, about 20 mm to about 26 mm, about 20 mm to about 24 mm, about 20 mm to about 22 nm, about 22 mm to about 30 mm, about 22 mm to about 28 mm, about 22 mm to about 26 mm, about 22 mm to about 24 mm, about 24 mm to about 30 mm, about 24 mm to about 28 mm, or about 24 mm to about 26 mm, and a width W of about 8 mm to about 16 mm, such as about 8 mm to about 14 mm, about 8 mm to about 12 mm, about 8 mm to about 10 mm, about 9 mm to about 16 mm, about 9 mm to about 14 mm, about 9 mm to about 12 mm, about 9 mm to about 10 mm, about 10 mm to about 16 mm, about 10 mm to about 14 mm, about 10 mm to about 12 mm, or about 14 to about 16. Certain example nano pouch products have dimensions of about 24 mm by about 9 mm, about 24 mm by about 8 mm, about 23 mm by about 9 nm, about 23 mm by about 8 mm, about 22 mm by about 9 mm, about 22 mm by about 9 mm, about 21 mm by about 9 mm, about 21 mm by about 8 mm, about 20 mm by about 9 mm or about 20 mm by about 8 mm.

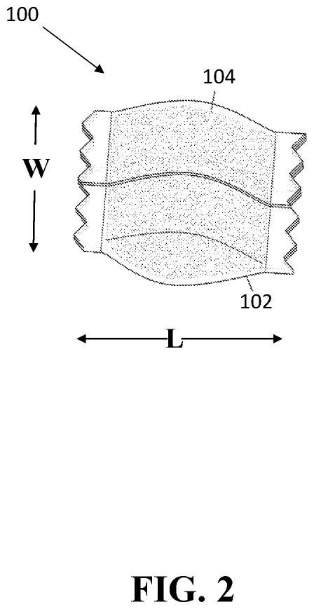

Certain specific example rectangular nano pouches are also provided herein, wherein L is equal to or close in value to W, such that the nano pouch is in the rough form of a square, as shown schematically in FIG. 2. Again, by the term “square” is intended not only a traditional square, with defined corners, but also square-like shapes with rounded edges (as referenced above with respect to “rectangular” pouched products. Example square nano pouches have length and width values that are both about 24 mm or less, about 22 mm or less, about 20 mm or less, about 18 mm or less, about 16 mm or less, about 14 mm or less, about 12 mm or less, or about 10 mm or less. In some such embodiments, a square nano pouch is provided with dimensions L≈W=about 8 mm, about 9 mm, about 10 mm, about 12 mm, about 14 mm, about 16 mm, about 18 mm, or about 20 mm.

Typically, the thickness of the disclosed pouched products (T, not shown in FIG. 1 or 2), understood to represent the 3-dimensional thickness of the products, is generally within the range of 2-8 mm or 5-10 mm, although the disclosure is not limited thereto. The disclosed pouched products generally comprise one or more seals that enclose and seal the material within the pouch, as shown in FIGS. 1 and 2 (the two substantially vertical lines at either end of the filled pouch, running along the width W). For the disclosed nano pouches, and as shown in FIGS. 1 and 2, the seals may be, for example, along two opposing sides to seal the opposing ends of the pouched product (referred to herein as “end” seals). Such nano pouches may further comprise a longitudinal seal, which can be thicker than the end seals, as it is on the rear side of the pouch and its size typically does not significantly affect mouthfeel of the product during use. FIGS. 1 and 2 show an exemplary longitudinal seal running through the center of the depicted pouched product from left to right (in the direction shown for “W”). It is noted that, in some embodiments, the end seals of the nano pouches provided herein are correspondingly smaller than in conventional products. In some embodiments, the nano pouches provided herein can have seals of about 1.5 mm or less. One very specific embodiment has dimensions of about 24 mm by about 9 mm, with end seals of about 1.5 mm.

In various embodiments, the total measurements for the length, width, and thickness (i.e., adding all four sides of the pouch, plus the thickness) are within the following ranges. In some embodiments, the total length, width, and thickness of a nano pouch as provided herein is about 130 mm or less, about 120 mm or less, about 110 mm or less, about 100 mm or less, about 90 mm or less, about 80 mm or less, about 70 mm or less, about 60 mm or less, about 50 mm or less, or about 40 mm or less, e.g., about 30 mm to about 130 mm, about 30 mm to about 100 mm, about 50 to about 100 mm, or about 50 to about 70 mm. Advantageously, in such embodiments, the thickness of such pouches is about 8 mm or less. Surface area of certain nano pouches (defined as length times width×2) is about 900 mm² or less, about 800 mm² or less, about 700 mm² or less, about 600 mm² or less, about 500 mm² or less, about 400 mm² or less, about 300 mm² or less, about 250 mm² or less, about 200 mm² or less, or about 150 mm² or less (e.g., with a minimum of about 100 mm²).

In some embodiments, such nano pouches can provide for faster release of the flavorant and/or active ingredient from the internal material to the consumer's oral cavity during use as compared with larger pouches (e.g., conventional pouches that are of analogous composition, but with larger dimensions). In certain embodiments, such nano pouches can provide for more comfort within the consumer's oral cavity, given their smaller size as compared with conventional pouched products. Such size can allow these products to be, in some embodiments, more readily accommodated at various positions within the oral cavity. Such smaller products also may, in some embodiments, allow for use to be more (as the user may, in some embodiments, readily “hide” the product, e.g., between his/her gum and lip).

It is noted that to provide such “nano pouch” products, which in various embodiments are smaller in one or more dimension than conventional pouched products, standard production equipment must be suitably modified, with different assemblies required for such low lengths and/or widths. Manufacturing apparatus and methods that can be adapted for preparation of nano pouches according to the present disclosure include, e.g., those disclosed in U.S. Patent Application Publication No. 2012/0055493 to Novak, III et al., incorporated herein by reference in its entirety, relates to an apparatus and process for providing pouch material formed into a tube for use in the manufacture of smokeless tobacco products. Similar apparatuses that incorporate equipment for supplying a continuous supply of a pouch material (e.g., a pouch processing unit adapted to supply a pouch material to a continuous tube forming unit for forming a continuous tubular member from the pouch material) can be used to create a pouched product described herein. Representative equipment for forming such a continuous tube of pouch material is disclosed, for example, in U.S. Patent Application Publication No. US 2010/0101588 to Boldrini et al., which is incorporated herein by reference in its entirety. The apparatus further includes equipment for supplying pouched material to the continuous tubular member such that, when the continuous tubular member is subdivided and sealed into discrete pouch portions, each pouch portion includes a charge of a composition adapted for oral use. Representative equipment for supplying the filler material is disclosed, for example, in U.S. Patent Application Publication No. US 2010/0018539 to Brinkley, which is incorporated herein by reference in its entirety. In some instances, the apparatus may include a subdividing unit for subdividing the continuous tubular member into individual pouch portions and, once subdivided into the individual pouch portions, may also include a sealing unit for sealing at least one of the ends of each pouch portion. In other instances, the continuous tubular member may be sealed into individual pouch portions with a sealing unit and then, once the individual pouch portions are sealed, the continuous tubular member may be subdivided into discrete individual pouch portions by a subdividing unit subdividing the continuous tubular member between the sealed ends of serially-disposed pouch portions. Still in other instances, sealing (closing) of the individual pouch portions of the continuous tubular member may occur substantially concurrently with the subdivision thereof, using a closing and dividing unit.

An example apparatus for manufacturing an oral pouch product, which, again, may be suitably modified as needed to produce the disclosed nano pouches, is illustrated in FIGS. 1-5 of U.S. Publication No. 2012/0055493 to Novak, III et al.; however, this apparatus is used in a generic and descriptive sense only and not for purposes of limitation. It should also be appreciated that the following manufacturing process and related equipment is not limited to the process order described below. In various embodiments of the present invention, an apparatus similar to that described in U.S. Patent Application Publication No. 2012/0055493 can be configured to removably receive a first bobbin on an unwind spindle assembly, the first bobbin having a continuous length of a material, such as a pouch material, wound thereon. When the first bobbin is engaged with the apparatus, the pouch material can be routed from the first bobbin to a forming unit configured to form a continuous supply of the pouch material into a continuous tubular member defining a longitudinal axis.

As such, as the pouch material is unwound from the first bobbin, the pouch material can be directed around an arrangement of roller members, otherwise referred to herein as a dancer assembly. A forming unit can be configured to cooperate with the first bobbin and the dancer assembly to take up slack in the pouch material and to maintain a certain amount of longitudinal tension on the pouch material as the pouch material is unwound from the first bobbin and fed to the forming unit, for example, by a drive system. One of ordinary skill in the art will appreciate that, between the first bobbin and the forming unit, the pouch material can be supported, routed, and/or guided by a suitably aligned series of any number of, for example, idler rollers, guideposts, air bars, turning bars, guides, tracks, tunnels, or the like, for directing the pouch material along the desired path. Typical bobbins used by conventional automated pouch making apparatuses often contain a continuous strip of pouch material of which the length may vary. As such, the apparatus described herein can be configured so as to handle bobbins of that type and size.

The forming unit can include one or more roller members configured to direct the pouch material about a hollow shaft such that the continuous supply of the pouch material can be formed into a continuous tubular member. The forming unit can include a sealing device configured to seal, fix, or otherwise engage lateral edges of the pouch material to form a longitudinally-extending seam, thereby forming a longitudinally-extending continuous tubular member. In various embodiments, an insertion unit can be configured to introduce charges of the composition adapted for oral use into the continuous tubular member through the hollow shaft. The insertion unit may be directly or indirectly engaged with the hollow shaft.

A leading edge or end (also referred to as a laterally-extending seam) of the continuous tubular member can be closed/sealed such that a charge of composition adapted for oral use inserted by the insertion unit, is contained within the continuous tubular member proximate to the leading end. The leading end can be closed/sealed via a closing and dividing unit configured to close/seal a first portion of the continuous tubular member to form the closed leading end of a pouch member portion. The closing and dividing unit can also be configured to form a closed trailing edge or end of a previous pouch member portion. In this regard, the closing and dividing unit can also be configured to close a second portion of the continuous tubular member to form the closed trailing end of the pouch member portion. In this regard, the closing and dividing unit can close the ends, by heat-sealing, or other suitable sealing mechanism.

As illustrated in FIGS. 20-22 of U.S. Publication No. 2012/0055493 to Novak, III et al., the closing and dividing unit can be configured to divide the continuous tubular member, between the closed trailing end and the closed leading end of serially-disposed pouch member portions, along the longitudinal axis of the continuous tubular member, and into a plurality of discrete pouch member portions such that each discrete pouch member portion includes a portion of the oral composition from the insertion unit. In this regard, the closing and dividing unit can include a blade, heated wire, or other cutting arrangement for severing the continuous tubular member into discrete pouch member portions. For example, the closing and dividing unit can include first and second arm members configured to interact to close and divide the continuous tubular member.

In operation, a charge of the composition adapted for oral use (i.e., an amount suitable for an individual pouch member portion) can be supplied to the pouch member portion by an insertion unit after a leading end has been closed, but prior to the closing of a trailing end. In various embodiments, after receiving the charge of the oral composition, the discrete individual pouch member portion can be formed by closing the trailing end and severing the closed pouch member portion from the continuous tubular member such that an individual pouched product is formed. In some embodiments, the feed tube diameter and/or the fleece width is modified to provide the disclosed nano pouches.

### Large Pouches

It is noted that the section herein above is focused specifically on nano pouches, which are smaller in size than conventional pouched oral products. The disclosure also provides large tobacco pouches, which are larger in size than conventional pouched oral products. Such large pouches may be particularly beneficial, e.g., to users who may use multiple pouches at a single time. In some embodiments, such large pouches may allow for more extended release of the active ingredient(s) in the composition contained within. Certain large pouches have a width of about 8 to about 18 mm and a length of about 35 to about 60 mm.

The sizing of certain large pouched products according to the present disclosure can be understood through reference to FIG. 1. Certain, non-limiting examples of rectangular large pouches provided herein are as follows: a large pouch with L≥35 mm and W≥8 mm, a large pouch with L≥35 mm and W≥10 mm, a large pouch with L≥35 mm and W≥12 mm, a large pouch with L≥35 mm and W≥14 mm, a large pouch with L≥35 mm and W≥16 mm, a large pouch with L≥40 mm and W≥8 mm, a large pouch with L≥40 mm and W≥10 mm, a large pouch with L≥40 mm and W≥12 mm, a large pouch with L≥40 mm and W≥14 mm, a large pouch with L≥40 mm and W≥16 mm, a large pouch with L≥50 mm and W≥8 mm, a large pouch with L≥50 mm and W≥10 mm, a large pouch with L≥50 mm and W≥12 mm, a large pouch with L≥50 mm and W≥14 mm, and a large pouch with L≥50 mm and W≥16 mm.

Certain advantageous ranges of length and width of such large pouches are, in some embodiments, a length L of about 35 mm to about 60 mm, such as about 40 mm to about 60 mm, about 50 mm to about 60 mm, about 35 mm to about 50 mm, and about 35 mm to about 40 mm, and a width W of about 8 mm to about 16 mm, such as about 8 mm to about 14 mm, about 8 mm to about 12 mm, about 8 mm to about 10 mm, about 9 mm to about 16 mm, about 9 mm to about 14 mm, about 9 mm to about 12 mm, about 9 mm to about 10 mm, about 10 mm to about 16 mm, about 10 mm to about 14 mm, about 10 mm to about 12 mm, or about 14 to about 16.

In various embodiments, the total measurements for the length, width, and thickness (i.e., adding all four sides of the pouch, plus the thickness) are within the following ranges. In some embodiments, the total length, width, and thickness of a nano pouch as provided herein is about 90 mm or greater, about 100 mm or greater, about 110 mm or greater, about 120 mm or greater, about 130 mm or greater, about 140 mm or greater, or about 150 mm or greater. Advantageously, in such embodiments, the thickness of such pouches is about 2 mm or greater (e.g., between about 2 and about 8 mm) Surface area of certain large pouches (defined as length times width×2) is about 300 mm² or greater, about 400 mm² or greater, about 500 mm² or greater, about 600 mm² or greater, or about 700 mm² or greater (e.g., with a maximum of about 1000 mm²).

In some embodiments, such large pouches can provide for slower release of the flavorant and/or active ingredient from the internal material to the consumer's oral cavity during use as compared with smaller pouches (e.g., conventional pouches that are of analogous composition, but with smaller dimensions). In certain embodiments, such large pouches can provide for greater user enjoyment, e.g., where the user has a larger oral cavity or prefers using multiple conventional pouches simultaneously, given their larger size as compared with conventional pouched products. In some embodiments, a larger pouch will allow for the inclusion of more material 102 within the pouch. Such additional material may comprise any of the types of components described herein; in some embodiments, the inclusion of more material 102 can involve the inclusion of greater amounts of active ingredient and/or greater amounts of flavorant than in conventional pouched products.

It is noted that, as with nano pouches, to provide such “large pouch” products (which in various embodiments are larger in one or more dimension than conventional pouched products), standard production equipment must be suitably modified, with different assemblies required for such large lengths and/or widths. Manufacturing apparatus and methods that can be adapted for preparation of large pouches according to the present disclosure include, e.g., those disclosed herein above in the context of nano pouches and in the referenced patent documents.

### Shaped Pouches

The disclosure provides, in additional embodiments, pouched products of shapes other than conventional rectangles and squares (as referenced herein above with respect to “conventional” pouched products). Such products are provided in varying shapes and sizes. Advantageously, by tailoring the shape of pouched products, the products may, in some embodiments, more readily be accommodated within a user's oral cavity. In other words, shaped pouches can be suitably designed to conform to a portion of the shape of the oral cavity of a user. For example, in some embodiments, shaped pouches are provided which more closely resemble the curve of a user's jaw and/or gumline, so as to increase the comfort within the oral cavity during use. In some embodiments, the disclosed shaped pouched products are described as being more comfortably accommodated or retained within the oral cavity during use than conventional pouched products.

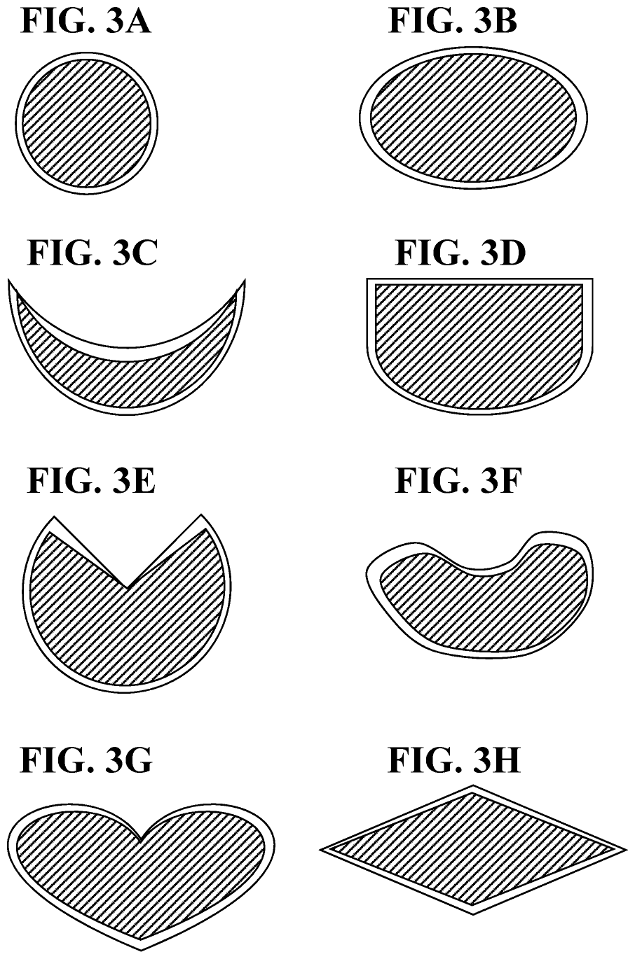

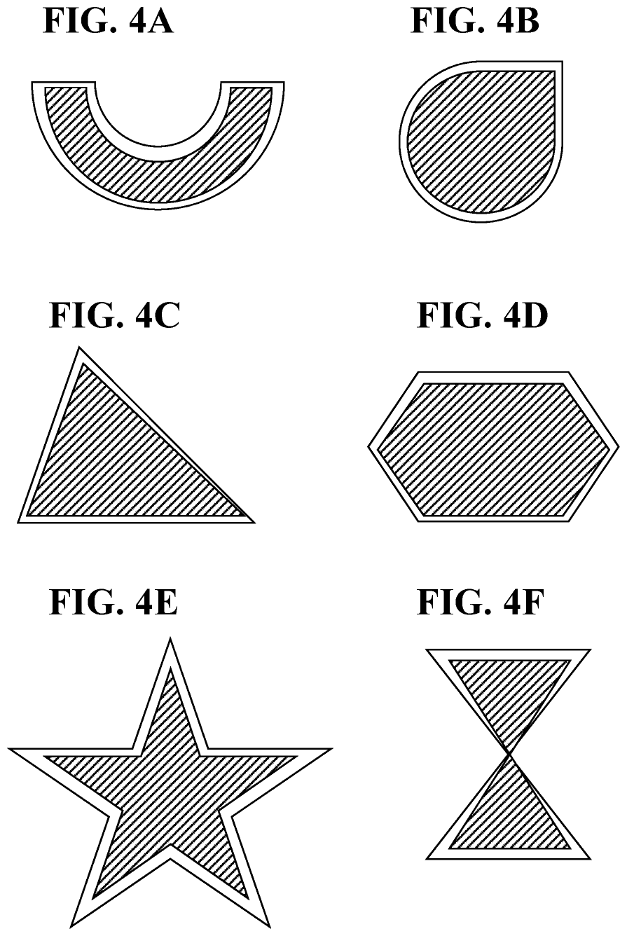

The exact shapes of pouches are not particularly limited. In certain preferred embodiments, shaped pouches provided herein comprise at least one rounded dimension/edge. Certain non-limiting shapes are provided in FIGS. 3A, 3B, 3C, 3D, 3E, 3F, 3G, 3H and FIGS. 4A, 4B, 4C, 4D, 4E, and 4F. Various shapes can be described, for example, as “circular,” “oval,” “oblong,” “crescent-shaped,” “rounded crescent-shaped,” “half-moon-shaped,” “half-circular,” “teardrop-like,” “star-shaped,” “domed,” “rhombic,” “rounded rhombic,” “diamond-shaped,” “rounded diamond-shaped,” “kidney-shaped,” “heart-shaped,” “triangular,” “rounded triangular” (including, e.g., isosceles, equilateral, scalene, acute, right, and obtuse) “hexagonal,” “rounded hexagonal” (including hexagonal with equal length edges and with varying length edges) and the like. The term “rounded” in such definitions refers to rounded edges (rather than the sharp edges shown in certain of the example shapes provided in FIGS. 3A, 3B, 3C, 3D, 3E, 3F, 3G, 3H, and FIGS. 4A, 4B, 4C, 4D, 4E, and 4F. Such shapes may be substantially uniform in thickness or may vary across the length or width of the pouched product, e.g., providing a three-dimensionally shaped structure such as a dome (with a higher center), or a cone-typed structure (e.g., with greater thickness at the bottom of a triangular or rounded triangular-type shape).

The sizes of the shaped pouched products provided herein can vary widely. In some embodiments, the shaped pouched products are designed so as to be substantially similar in size to conventional pouched products. In other embodiments, they may be somewhat larger in size or somewhat smaller in size. Again, in some embodiments, consistent with the noted advantage of certain shaped “nano” products being designed to conform to a user's jaw or gumline, the pouched product may be sized accordingly to fit and be maintained in the desired position within the oral cavity. In some embodiments, a pouched product can be provided which is considered both a “shaped pouch” according to this disclosure, and also a “nano pouch” according to this disclosure. In further embodiments, a pouched product can be provided which is considered both a “shaped pouch” according to this disclosure, and also a “large pouch” according to this disclosure.

It is noted that to provide such shaped pouch products, which are shaped differently than conventional pouched products, standard production equipment and/or processes (as referenced herein above) must be modified accordingly, with different assemblies required for, e.g., curved sides and alternative angles of sides. For example, in some embodiments, an appropriately sized/shaped assembly (e.g., a heated shaped cutter) is employed to provide the desired shapes. For example, in one specific embodiment, the assembly may be pressed against an anvil with a filled tube of fleece and filler passing between them.

### Components of the Disclosed Nano Pouches, Large Pouches, and Shaped Pouches

The disclosure thus far has focused on the general overall size and/or shape of the exterior of pouched products. Pouched products generally comprise, in addition to the pouch-based exterior, a mixture within the pouch that typically comprises one or more active ingredients and/or one or more flavorants, and various other optional ingredients. The composition of the material within the nano pouches, large pouches, and shaped pouches provided herein is not particularly limited, and can comprise any filling composition, including those included within conventional pouched produces. Such compositions are generally mixtures of two or more components and as such, the compositions are, in some cases, referenced herein below as “mixtures.” Certain components that can advantageously be included in the mixtures within certain embodiments of the nano pouches, large pouches, and shaped pouches provided herein are outlined generally below; however, it is to be understood that the discussion below is not intended to be limiting of the components that can be incorporated within the disclosed nano pouches, large pouches, and shaped pouches. In some embodiments, the weight of the mixture within each pouch is at least about 50 mg, for example, from about 50 mg to about 2 grams, from about 100 mg to about 1.5 grams, or from about 200 to about 700 mg. For example, in the smaller (e.g., nano) pouches, less of the mixture is generally contained than in the larger pouches. In certain embodiments, smaller pouches (which may be, e.g., nano pouches and/or shaped pouches) may contain, e.g., about 50 mg to about 600 mg, about 50 mg to about 500 mg, about 50 mg to about 400 mg, about 50 mg to about 300 mg, about 50 mg to about 200 mg, or about 50 mg to about 100 mg of the mixture. In some embodiments, pouches (which can, in various embodiments, be considered nano pouches or large pouches and which can, in some embodiments, be shaped pouches), may contain, e.g., about 500 mg to about 1 gram, about 500 mg to about 800 mg, about 600 mg to about 1 gram, about 600 mg to about 800 mg, or about 800 mg to about 1 gram of the mixture. Further pouches (which may be, e.g., large pouches and/or shaped pouches) may contain about 800 mg to about 2 grams, about 800 mg to about 1.5 grams, about 800 mg to about 1.2 grams, about 1 gram to about 2 grams, about 1.5 grams to about 2 grams, or about 1 gram to about 1.5 grams of the mixture.

### Filler

The material within the pouches as described herein typically includes at least one particulate filler. Such particulate fillers may fulfill multiple functions, such as enhancing certain organoleptic properties such as texture and mouthfeel, enhancing cohesiveness or compressibility of the product, and the like. Generally, the fillers are porous particulate materials and are cellulose-based. For example, suitable particulate fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate fillers include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, the particulate filler is a cellulose material or cellulose derivative. One particularly suitable particulate filler for use in the products described herein is microcrystalline cellulose (“MCC”). The MCC may be synthetic or semi-synthetic, or it may be obtained entirely from natural celluloses. The MCC may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, the mixture comprises MCC as the particulate filler. The quantity of MCC present in the mixture as described herein may vary according to the desired properties.

The amount of particulate filler can vary, but is typically up to about 75 percent of the material contained within the pouch by weight (i.e., the mixture), based on the total weight of the mixture. A typical range of particulate filler material (e.g., MCC) within the mixture can be from about 10 to about 75 percent by total weight of the mixture, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of particulate filler material is at least about 10 percent by weight, such as at least about 20 percent, or at least about 25 percent, or at least about 30 percent, or at least about 35 percent, or at least about 40 percent, based on the total weight of the mixture.

In one embodiment, the particulate filler further comprises a cellulose derivative or a combination of such derivatives. In some embodiments, the mixture comprises from about 1 to about 10% of the cellulose derivative by weight, based on the total weight of the mixture, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In some embodiments, the mixture comprises from about 1 to about 3% HPC by weight, based on the total weight of the mixture.

### Water

The water content of the mixture within the pouched product described herein, prior to use by a consumer of the product, may vary according to the desired properties. Typically, the mixture, as present within the product prior to insertion into the mouth of the user, is less than about 60 percent by weight of water, and generally is from about 1 to about 60% by weight of water, for example, from about 5 to about 55, about 10 to about 50, about 20 to about 45, or about 25 to about 40 percent water by weight, including water amounts of at least about 5% by weight, at least about 10% by weight, at least about 15% by weight, and at least about 20% by weight.

### Flavoring Agent

As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, trigeminal sensates, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin

The amount of flavoring agent utilized in the mixture can vary, but is typically up to about 10 weight percent, and certain embodiments are characterized by a flavoring agent content of at least about 0.1 weight percent, such as about 0.5 to about 10 weight percent, about 1 to about 6 weight percent, or about 2 to about 5 weight percent, based on the total weight of the mixture.

The amount of flavoring agent present within the mixture may vary over a period of time (e.g., during a period of storage after preparation of the mixture). For example, certain volatile components present in the mixture may evaporate or undergo chemical transformations, leading to a reduction in the concentration of one or more volatile flavor components. In one embodiment, a concentration of one or more of the at least one volatile flavor components present is greater than a concentration of the same one or more volatile flavor components present in a control pouched product which does not include the one or more organic acids, after the same time period. Without wishing to be bound by theory, it is believed that the same mechanisms responsible for loss of whiteness result in a gradual decline in certain volatile components in the flavoring (e.g., aldehydes, ketones, terpenes). Therefore, a decline in the presence of these volatile components leading to the discoloration over time may be expected to diminish the sensory satisfaction associated with products subject to such a degradation process.

### Salts

In some embodiments, the mixture may further comprise a salt (e.g., alkali metal salts), typically employed in an amount sufficient to provide desired sensory attributes to the mixture. Non-limiting examples of suitable salts include sodium chloride, potassium chloride, ammonium chloride, flour salt, and the like. When present, a representative amount of salt is about 0.5 percent by weight or more, about 1.0 percent by weight or more, or at about 1.5 percent by weight or more, but will typically make up about 10 percent or less of the total weight of the mixture, or about 7.5 percent or less or about 5 percent or less (e.g., about 0.5 to about 5 percent by weight).

### Sweeteners

The mixture typically further comprises one or more sweeteners. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, isomaltulose stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). When present, a representative amount of sweetener may make up from about 0.1 to about 20 percent or more of the of the mixture by weight, for example, from about 0.1 to about 1%, from about 1 to about 5%, from about 5 to about 10%, or from about 10 to about 20% of the mixture on a weight basis, based on the total weight of the mixture.

### Binding Agents

A binder (or combination of binders) may be employed in certain embodiments, in amounts sufficient to provide the desired physical attributes and physical integrity to the mixture, and binders also often function as thickening or gelling agents. Typical binders can be organic or inorganic, or a combination thereof. Representative binders include povidone, sodium alginate, starch-based binders, pectin, carrageenan, pullulan, zein, and the like, and combinations thereof. In some embodiments, the binder comprises pectin or carrageenan or combinations thereof.

The amount of binder utilized in the mixture can vary, e.g., based on the binder and the desired mixture properties, but is typically up to about 30 weight percent, and certain embodiments are characterized by a binder content of at least about 0.1% by weight, such as about 1 to about 30% by weight, or about 5 to about 10% by weight, based on the total weight of the mixture.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the mixture.

### Humectants

In certain embodiments, one or more humectants may be employed in the mixture. Examples of humectants include, but are not limited to, glycerin, propylene glycol, and the like. Where included, the humectant is typically provided in an amount sufficient to provide desired moisture attributes to the mixture. Further, in some instances, the humectant may impart desirable flow characteristics to the mixture for depositing in a mold. When present, a humectant will typically make up about 5% or less of the weight of the mixture (e.g., from about 0.5 to about 5% by weight). When present, a representative amount of humectant is about 0.1% to about 1% by weight, or about 1% to about 5% by weight, based on the total weight of the mixture.

### Buffering Agents

In certain embodiments, the mixture of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the mixture, for example, from about 0.5% to about 5%, such as, e.g., from about 0.75% to about 4%, from about 0.75% to about 3%, or from about 1% to about 2% by weight, based on the total weight of the mixture. Non-limiting examples of suitable buffers include alkali metals acetates, glycinates, phosphates, glycerophosphates, citrates, carbonates, hydrogen carbonates, borates, or mixtures thereof.

### Colorants

A colorant may be employed in amounts sufficient to provide the desired physical attributes to the mixture. Examples of colorants include various dyes and pigments, such as caramel coloring and titanium dioxide. The amount of colorant utilized in the mixture can vary, but when present is typically up to about 3 weight percent, such as from about 0.1%, about 0.5%, or about 1%, to about 3% by weight, based on the total weight of the mixture.

### Active Ingredient

The mixture may additionally include one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the mixture, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the mixture. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the mixture. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the mixture.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the mixture.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the mixture.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the mixture. In some embodiments, the composition comprises caffeine in an amount of from about 1.5 to about 6% by weight, based on the total weight of the mixture.

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the mixture.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinol-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the mixture.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the mixture.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride. In some embodiments, the nicotine component or a portion thereof is a nicotine salt with one or more organic acids.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the mixture, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the mixture. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the mixture. These ranges can also apply to other active ingredients noted herein.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Tobacco Material

In some embodiments, the mixture may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N.×sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; 5,387,416 to White et al., U.S. Pat. No. 7,025,066 to Lawson et al.; U.S. Pat. No. 7,798,153 to Lawrence, Jr. and 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; 5,387,416 to White et al.; and 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina) The mixture disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the d mixture optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the mixture for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int, 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int, 21, 305-320 (2005) and Staaf et al., Beitrage Tabakforsch. Int, 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rusfica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. No. 787,611 to Daniels, Jr.; U.S. Pat. No. 1,086,306 to Oelenheinz; U.S. Pat. No. 1,437,095 to Delling; U.S. Pat. No. 1,757,477 to Rosenhoch; U.S. Pat. No. 2,122,421 to Hawkinson; U.S. Pat. No. 2,148,147 to Baier; U.S. Pat. No. 2,170,107 to Baier; U.S. Pat. No. 2,274,649 to Baier; U.S. Pat. No. 2,770,239 to Prats et al.; U.S. Pat. No. 3,612,065 to Rosen; U.S. Pat. No. 3,851,653 to Rosen; U.S. Pat. No. 3,889,689 to Rosen; U.S. Pat. No. 3,943,940 to Minami; U.S. Pat. No. 3,943,945 to Rosen; U.S. Pat. No. 4,143,666 to Rainer; U.S. Pat. No. 4,194,514 to Campbell; U.S. Pat. Nos. 4,366,823, 4,366,824, and 4,388,933 to Rainer et al.; U.S. Pat. No. 4,641,667 to Schmekel et al.; U.S. Pat. No. 5,713,376 to Berger; U.S. Pat. No. 9,339,058 to Byrd Jr. et al.; 9,420,825 to Beeson et al.; and 9,950,858 to Byrd Jr. et al.; as well as in US Pat. App. Pub. Nos. 2012/0067361 to Bjorkholm et al.; 2016/0073686 to Crooks; 2017/0020183 to Bjorkholm; and 2017/0112183 to Bjorkholm, and in PCT Publ. Appl. Nos. WO1996/031255 to Giolvas and WO2018/083114 to Bjorkholm, all of which are incorporated herein by reference.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and 7,337,782 to Thompson, all of which are incorporated by reference herein.

Typical inclusion ranges for tobacco materials can vary depending on the nature and type of the tobacco material, and the intended effect on the final mixture, with an example range of up to about 30% by weight (or up to about 20% by weight or up to about 10% by weight or up to about 5% by weight), based on total weight of the mixture (e.g., about 0.1 to about 15% by weight). In some embodiments, the products of the disclosure can be characterized as completely free or substantially free of tobacco material (other than purified nicotine as an active ingredient). For example, certain embodiments can be characterized as having less than 1% by weight, or less than 0.5% by weight, or less than 0.1% by weight of tobacco material, or 0% by weight of tobacco material.

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the mixture.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the mixture.

In some embodiments, the mixture is substantially free of any API. By “substantially free of any API” means that the composition does not contain, and specifically excludes, the presence of any API as defined herein, such as any Food and Drug Administration (FDA) approved therapeutic agent intended to treat any medical condition.

### Other Additives

Other additives can be included in the disclosed mixture. For example, the mixture can be processed, blended, formulated, combined and/or mixed with other materials or ingredients. The additives can be artificial, or can be obtained or derived from herbal or biological sources. Examples of further types of additives include thickening or gelling agents (e.g., fish gelatin), emulsifiers, oral care additives (e.g., thyme oil, eucalyptus oil, and zinc), preservatives (e.g., potassium sorbate and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final mixture, with an example range of up to about 10% by weight, based on total weight of the mixture (e.g., about 0.1 to about 5% by weight).

The aforementioned additives can be employed together (e.g., as additive formulations) or separately (e.g., individual additive components can be added at different stages involved in the preparation of the final mixture). Furthermore, the aforementioned types of additives may be encapsulated as provided in the final product or mixture. Example encapsulated additives are described, for example, in WO2010/132444 to Atchley, which has been previously incorporated by reference herein.

In some embodiments, any one or more of a filler, a tobacco material, and the overall oral product described herein can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre-determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, such as 300 μm.

In some embodiments, any particulate material referenced herein (e.g., filler, tobacco material, and the overall oral product) can be characterized as having at least 50% by weight of particles with a particle size as measured by sieve analysis of no greater than about 1000 μm, such as no greater than about 500 μm, such as no greater than about 400 μm, such as no greater than about 350 μm, such as no greater than about 300 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

### Preparation of the Mixture

The manner by which the various components of the mixture are combined may vary. As such, the overall mixture of various components with e.g., powdered mixture components may be relatively uniform in nature. The components noted above, which may be in liquid or dry solid form, can be admixed in a pretreatment step prior to mixture with any remaining components of the mixture, or simply mixed together with all other liquid or dry ingredients. The various components of the mixture may be contacted, combined, or mixed together using any mixing technique or equipment known in the art. Any mixing method that brings the mixture ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and 6,834,654 to Williams, each of which is incorporated herein by reference. In some embodiments, the components forming the mixture are prepared such that the mixture thereof may be used in a starch molding process for forming the mixture. Manners and methods for formulating mixtures will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and 6,077,524 to Bolder et al., each of which is incorporated herein by reference.

### Method of Improving Mouthfeel of a Pouched Product

In another aspect is provided a method of providing an oral product with improved mouthfeel (e.g., relative to conventional oral pouched products). Generally, the method comprises providing a nano pouch or a shaped pouch as provided herein which, in some embodiments, more closely approximates the shape or more readily conforms to at least a portion of the consumer's oral cavity (e.g., the consumer's jaw, gumline, etc.).

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A pouched product for oral use, comprising a material within a porous pouch,
wherein the material comprises one or more flavoring agents and/or one or more active ingredients;
wherein the porous pouch comprises four sides, and wherein none of the four sides of the porous pouch is more than 24 mm in length.

2. The pouched product of claim 1, wherein none of the four sides of the porous pouch is more than 22 mm in length.

3. The pouched product of claim 1, wherein none of the four sides of the porous pouch is more than 20 mm in length.

4. The pouched product of claim 1, wherein the four sides have lengths of about 24 mm, about 24 mm, about 9 mm, and about 9 mm.

5. The pouched product of claim 1, wherein the four sides have lengths that are substantially the same.

6. The pouched product of claim 5, wherein the four sides have lengths of about 16 mm or less.

7. The pouched product of any of claims 1 to 6, wherein the material further comprises one or more particulate fillers and water.

8. The pouched product of claim 7, wherein the one or more particulate fillers comprise a cellulose material.

9. The pouched product of claim 8, wherein the cellulose material comprises microcrystalline cellulose.

10. The pouched product of claim 7, wherein the one or more particulate fillers comprise a cellulose derivative in an amount by weight of the material of from about 1% to about 3%.

11. The pouched product of claim 1, wherein the one or more active ingredients are selected from the group consisting of a nicotine component, a nutraceutical, a botanical, a stimulant, an amino acid, a vitamin, a cannabinoid, a cannabimimetic, a terpene, and combinations thereof.

12.-16. (canceled)

17. A pouched product for oral use, comprising a material within a porous pouch,
wherein the material comprises one or more flavoring agents and/or one or more active ingredients;
wherein the porous pouch is in a shape that is not square or rectangular.

18. The pouched product of claim 17, wherein the shape is selected from the group consisting of triangular, rhombic, pentagonal, hexagonal, heptagonal, and octagonal.

19. The pouched product of claim 17, wherein the shape has one or more rounded sides.

20. The pouched product of claim 17, wherein the shape is selected from the group consisting of circular, semi-circular, oval, semi-oval, kidney-shaped, teardrop-shaped, star-shaped, heart-shaped, and crescent-shaped.

21.-30. (canceled)

31. A method of delivering one or more flavoring agents and/or one or more active ingredients, comprising providing the one or more flavoring agents and/or one or more active ingredients in the form of a material within a porous pouch,
wherein the porous pouch comprises four sides, and wherein none of the four sides of the porous pouch is more than 24 mm in length; or
wherein the porous pouch is in a shape that is not square or rectangular.
